(19) United States
(12) Patent Application Publication
(10) Pub. No.: US 2019/0110999 A1
(43) Pub. Date: Apr. 18, 2019

(54) PREVENTION OF LIPID ABSORPTION USING ARTIFICIAL LIPID SEQUESTRATION DEVICES

(71) Applicant: LIPIQUESTER LLC, Rochester, MN (US)

(72) Inventor: Jacob Jennings Orme, Rochester, MN (US)

(21) Appl. No.: 15/783,881

(22) Filed: Oct. 13, 2017

(51) Int. Cl. A61K 9/48

(52) U.S. Cl. CPC A61K 9/4866; A61K 9/4808

(57) ABSTRACT

An artificial device, method and system are provided for the manufacture and use of artificial nanospheres, enclosed resins, and similar technologies for sequestering lipophilic molecules from environments, such as a gastrointestinal tract.

### BACKGROUND

### Technical Field

This disclosure is related generally to nutrition, nutritional supplementation, the prevention of dietary absorption, and/or the prevention of side-effects relating to the prevention of dietary absorption using novel lipid sequestration methods.

### State of the Art

More than two thirds of adults in the United States are overweight, with similar trends throughout the world. This trend has two root causes: increased calorie intake and decreased calorie expenditure. These root causes are interrelated and mutually reinforcing. The result of these mutual effects is a spectrum of metabolic disease that includes diabetes, peripheral vascular disease, and heart disease. These diseases kill over 400,000 people in the United States each year, making up over a quarter of all causes of mortality.

Many efforts have been made to stem the tide of metabolic spectrum disease by targeting one of the two root causes explained above. Increased energy expenditure, while effective, has been difficult to maintain in most individuals. Similarly, diets restricting and altering food intake have shown promise but are challenging to maintain. Indeed, some diets that severely restrict carbohydrates (one of three primary sources of calories, the other two being proteins and fats/lipids) have led to higher mortality in some studies. No artificial dietary intervention has been shown to effectively reduce fat intake without untenable side-effects.

### SUMMARY

An aspect of the present disclosure includes an artificial manufactured lipophilic molecule sequestration device comprising: one or more biocompatible exteriors, and one or more lipophilic interiors, wherein the artificial manufactured lipophilic molecule sequestration device is configured for introduction into a gastrointestinal environment.

Another aspect of the present disclosure includes a method for manufacturing a plurality of artificial lipophilic molecule sequestration devices, the method comprising: combining one or more polymers in one or more solvents; adding polymerizing agents; and producing at least one artificial lipophilic molecule sequestration device; wherein the at least one artificial lipophilic molecule sequestration device is configured to sequester lipids from a gastrointestinal environment.

Still another aspect of the present disclosure includes method for lipid sequestration comprising: introducing, into a lipid-containing environment, a plurality of artificial manufactured lipophilic molecule sequestration devices, each of the plurality of artificial manufactured lipophilic molecule sequestration devices comprising: one or more biocompatible exteriors; and one or more lipophilic interiors; wherein the plurality of artificial manufactured lipophilic molecule sequestration devices sequester lipophilic molecules to reduce interaction with the lipid-containing environment.

Yet another aspect of the present disclosure includes a lipid sequestration system comprising: a plurality of artificial manufactured lipophilic molecule sequestration devices comprising: one or more biocompatible exteriors, and one or more lipophilic interiors; and an environment comprising a plurality of lipophilic molecules; wherein at least one artificial manufactured lipophilic molecule sequestration device of the plurality of artificial manufactured lipophilic molecule sequestration devices absorbs and sequesters at least one or more lipophilic molecule(s) of the plurality of lipophilic molecules to reduce interaction between the one or more lipophilic molecule(s) and the environment.

The foregoing and other features, advantages, and construction of the present disclosure will be more readily apparent and fully appreciated from the following more detailed description of the particular embodiments, taken in conjunction with the accompanying drawings.

### BRIEF DESCRIPTION OF THE DRAWINGS

Some of the embodiments will be described in detail, with reference to the following figures, wherein like designations denote like members:

FIG. 1 illustrates an embodiment of an artificial manufactured lipophilic molecule sequestration device comprising one or more biocompatible exteriors and one or more lipophilic interiors with one or more pores, according to particular embodiments;

FIG. 2 illustrates another embodiment of an artificial manufactured lipophilic molecule sequestration device comprising one or more biocompatible exteriors and one or more lipophilic interiors, according to particular embodiments;

FIG. 3 shows further embodiment of an artificial manufactured lipophilic molecule sequestration device including an enclosed resin comprising one or more biocompatible exteriors and one or more lipophilic interiors, according to some embodiments;

FIG. 4 show still another embodiment of an artificial manufactured lipophilic molecule sequestration device comprising one or more biocompatible exteriors and one or more lipophilic interiors with one or more pores, according to one particular embodiment;

FIG. 5 shows an example system in which one or more artificial manufactured lipophilic molecule sequestration devices are introduced into a body and pass through an environment such as a gastrointestinal tract, according to some embodiments;

FIG. 6 shows an example system in which one or more introduced artificial manufactured lipophilic molecule sequestration devices prevent the interaction of sequestered materials with an environment that comprises a portion of a gastrointestinal tract, substances, bacteria, or any combination thereof, according to some embodiments;

FIG. 7 shows an example embodiment in which embodiments of artificial manufactured lipophilic molecule sequestration devices are located within a fecal environment;

FIG. 8 shows an illustrative schematic of an embodiment of a method of manufacture for producing one or more artificial manufactured lipophilic molecule sequestration devices comprising a biocompatible exterior and a lipophilic interior, according to some embodiments; and,

FIG. 9 shows a further schematic illustrating an example method of manufacture for producing one or more artificial manufactured lipophilic molecule sequestration devices, according to some embodiments.

### DETAILED DESCRIPTION OF EMBODIMENTS

Ineffective attempts at calorie restriction have been made with various and different technologies. Replacement fats, for instance, taste like regular fats with similar mouthfeel but are not metabolized by the body; rather, they are excreted in the feces. Unfortunately, these replacement fats often cause major side-effects include diarrhea, steatorrhea, bloating, abdominal pain, gas, bacterial overgrowth, and/or anal leakage, which makes them unusable. Pharmaceutical inhibitors (e.g. enzyme inhibitors) of fat breakdown have been similarly ineffective. These prevent fat breakdown and absorption in the intestinal tract but, like replacement fats, are associated with side effects of intestinal gas, anal leakage, incontinence, bloating, nausea/vomiting, and pain. In addition, these inhibitors can cause jaundice, cold/flu symptoms, kidney stones, liver disease, and rashes. Other attempts to restrict calories and cholesterol has been the development of binding resins such as cholestyramine. Like the previous, these resins are associated with side effects of intestinal gas, anal leakage, etc as well as with side effects from systemic absorption and interaction. Still other attempts to restrict fat intake have included gastric bypass and similar surgeries or procedures. Such interventions often lead to bowel obstruction, dumping syndrome, gallstones, hernias, malnutrition, perforations, ulcers, infections, and other side-effects and adverse events.

An artificial device, method and system are provided for the manufacture and use of artificial nanoparticles, nanocapsules, enclosed resins, and similar technologies for sequestering lipophilic molecules, for use in the gastrointestinal tract, herein referred to as “artificial manufactured lipophilic molecule sequestration devices,” “nanosphere lipid sequestration devices,” or similar terms. The disclosed methods, systems, and device embodiments may have the unexpected benefit of preventing absorption, uptake, and/or deleterious effects of lipids in a body. Some embodiments may include steric, temporal, permeability-based, or other methods of specific capture of certain substances within or exclusion of certain substances from sequestration based on physical characteristics. Some embodiments may include methods of supplementation to prevent loss of fat-soluble vitamins or other nutrients or to enhance removal of specific deleterious materials. Still further embodiments may include features for the restriction of, removal of, and/or toxicity to bacteria and/or other organisms.

In contrast with prior technologies, unexpected benefits of some embodiments disclosed herein may be accomplished without causing major side-effects including but not limited to diarrhea, steatorrhea, bloating, abdominal pain, gas, bacterial overgrowth, and anal leakage. Such benefits may stem from pores in certain specific embodiments that exclude bacteria through one or more mechanisms described. Such benefits may otherwise stem from biocompatible exteriors of specific embodiments that interact favorably with the environment of the intestinal tract. Examples of such favorable interactions may include preventing absorption, increasing the bulk of the stool, preventing constipation or motility issues, and/or other effects. Other additional potential benefits of some embodiments may include but are not limited to increased exercise tolerance, weight loss, improved psychosocial functioning, and improved functioning in activities of daily living. Some further embodiments may be designed to improve nutrition, augment health, or increase wellbeing or comfort. Such benefits may stem from prevention of interaction of unabsorbed fats with the gastrointestinal environment, which may comprise the gastrointestinal wall, bacteria, other organisms, and other substances. Some benefits may stem from various features and the present disclosures, embodiments, and benefits should not be considered limiting.

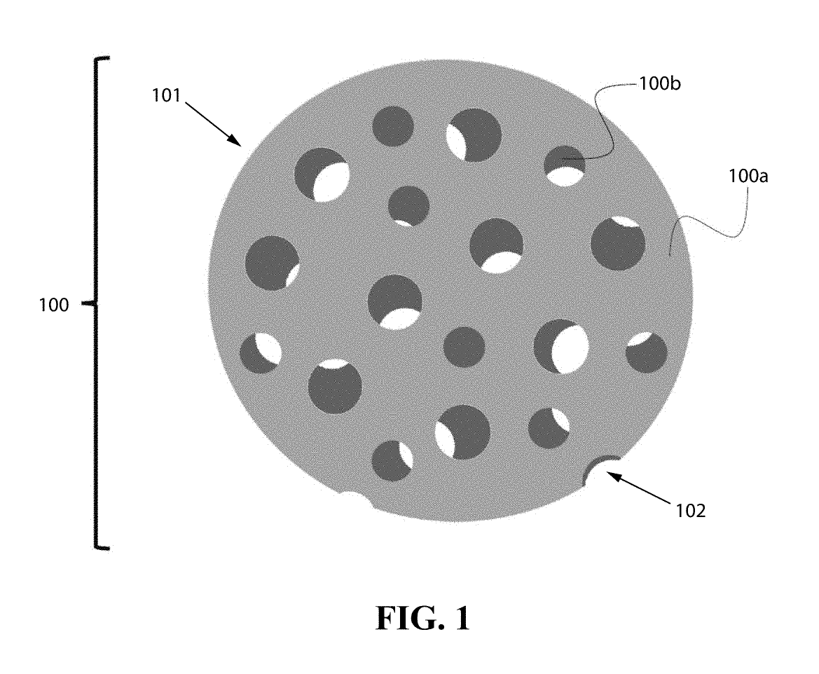

The provision of FIGS. 1 through 9, discussed herein below, and the various embodiments used to describe the principles of the present disclosure are by way of illustration only and should not be construed in any way to limit the scope of the disclosure. Those with ordinary skill in the art will understand that the principles of the present disclosure may be implemented in any suitably arranged configuration. The present disclosure may, however, be embodied in many different forms and should not be construed as limited to the embodiments set forth herein. Rather, the disclosed embodiments are provided such that this disclosure will be thorough and complete, and will convey the scope of the disclosure to those with ordinary skill in the art. The principles and features of the disclosure may be employed in varied and numerous embodiments without departing from the scope of the disclosure.

As depicted in the drawings, FIG. 1 shows an exemplary artificial manufactured lipophilic molecule sequestration device 100 including a nanosphere 101 comprising one or more biocompatible exteriors 100a and one or more lipophilic interiors 100b, according to one embodiment. The term “nanosphere” and similar terms such as “nanocapsule,” and “nanodevice,” as used herein, may refer to any sub-centimeter-sized spherical, semi-spherical, cubic, 3-dimensional, and/or globular object that may contain or be made to contain a substance. The term “artificial,” as used herein, means non-natural and/or made by man. The term “biocompatible,” as used herein and understood by those with ordinary skill in the art, refers to any substance that interacts with intestinal walls and/or luminal contents with an acceptable (i.e. low) level of toxicity for biological use. According to some embodiments, a biocompatible exterior 100a is hydrophilic. This may have the unexpected advantage of improving gut motility and otherwise behaving like fiber in the diet.

The terms “lipophilic,” “fats,” and related terms used herein refer to substances such as lipids, fats, fatty acids, triglycerides, diglycerides, monoglycerides, steroids, oils, enzymes, resins, fatty acid-interacting, fat-soluble, hydrophobic, lipophilic, aliphatic, aromatic, enzymatic, organic, and/or otherwise mutual fat-interacting materials that mutually attract or interact preferentially as understood by those with skill in the art. The term lipophilic and related terms may refer to an embodiment itself, a part of an embodiment, lipids or other substances with which embodiments interact, or other materials and should thus not be considered limiting for the purposes of this disclosure.

Some embodiments of artificial manufactured lipophilic molecule sequestration devices 100 are configured to include a functional a lipophilic interior 100b to sequester dietary fats, fatty acids, lipids, triglycerides, steroids, oils, and/or other lipophilic materials. “Sequester,” “sequestration,” and related terms, as used herein, refer to the separation of contained objects from non-contained objects or environments, wherein the environments may contain lipophilic materials, such as lipids, lipophilic molecules, dietary fats, fatty acids, lipids, triglycerides, steroids, oils, and other like materials. In some embodiments, a contained object comprises some combination of lipophilic molecules such as fats, fatty acids, triglycerides, oils, a combination thereof, etc. The containment of these lipophilic molecules may, for instance, separate (e.g. sequester) them from bacteria. Moreover, in some embodiments, this sequestration may have the unexpected benefit of preventing interaction of the contained object with an environment, such as a lipid-containing environment. In addition, some embodiments accomplish sequestration of or from lipids, other lipophilic substances, proteins, carbohydrates, enzymes, cells, luminal contents, luminal walls, bacteria, other organisms, chemicals, gastrointestinal environments, or any association or combination of any number of the above.

With further reference to FIG. 1, in some embodiments, the biocompatible exterior 100a is hydrophilic. The term “hydrophilic” as used herein refers to any substance that interacts preferentially with water due to its polar nature as understood by those with ordinary skill in the art. An unexpected experimental result of artificial manufactured lipophilic molecule sequestration devices with hydrophilic exteriors is their bulking effect in the intestines analogous to that of added dietary fibers. In some embodiments, this may have the beneficial result of reduced constipation, reduced diarrhea, and normalized bowel movements; decreased risk of diverticulosis, colon cancer, and other maladies; improved gut motility and reduced bacterial growth; and lower cholesterol levels, controlled blood sugar levels, and improved weight.

Embodiments of an artificial manufactured lipophilic molecule sequestration device 100 may include a nanosphere 101 having one or more pores 102 that allow entry of substances for sequestration. In some embodiments, these pores 102 are sufficiently large such that some substances preferentially permeate the exterior. For instance, in some embodiments, pores have an opening, greater than 7 angstroms (Å) in in diameter such that free fatty acids may enter freely. In still other embodiments, pores 102 allow entry to desired substances by size, charge, and/or shape. In, yet, still other embodiments, these pores are small such that some substances do not permeate the exterior. For instance, in some embodiments pores 102 have an opening smaller than 100 Å, such that bacteria (with diameters generally greater than 100 Å) cannot penetrate the structure. An unexpected benefit of restricted pore size may be the selective exclusion of bacteria based on size. For example, if a particular bacterium is larger than the pore size, the bacterium is excluded from entering via the pore because the bacterium is too large to enter easily. In still other embodiments, pores 102 exclude unwanted substances other than bacteria from the embodiment by size. Such substances may include, for instance, enzymes, fungi, protozoa, proteins, drugs, organisms, villi, extensions of the intestinal tract, cells, blood, and/or other substances. Moreover, in other embodiments, pores exclude unwanted substances by charge. In one such embodiment, highly negative pore edges exclude anions from penetration of the structure. Such an anion may include bacteria, albumin, or other substances. Furthermore, in still other embodiments, pores 102 may be opened or closed according to environment. For example, pores 102 may be closed in an acidic or positively-charged environment but open in a less acidic or less positively-charged environment using methods known to those of ordinary skill in the art. Embodied pores 102 may open, close, or include specificity based on chemical makeup.

Some embodiments of a nanosphere 101 may include pores, channels, and/or matrices 102 that allow entry and sequestration of materials. In some embodiments, these pores, channels, or matrices 102 exclude unwanted substances by size, shape, charge, solubility, and/or other property. Additionally, in some embodiments, these pores, channels, or matrices 102 create a barrier to entry to unwanted substances by size, shape, charge, and/or other property. In still other embodiments, pores, channels, or matrices 102 exclude unwanted substances by charge. Moreover, in other embodiments, pores, channels, or matrices 102 may be opened or closed according to environment. For instance, some pores 102 may include protein—associated electrical charges that change with environment such that more acidic conditions open the pores and more basic conditions close the pores 102 as understood by those of ordinary skill in the art.

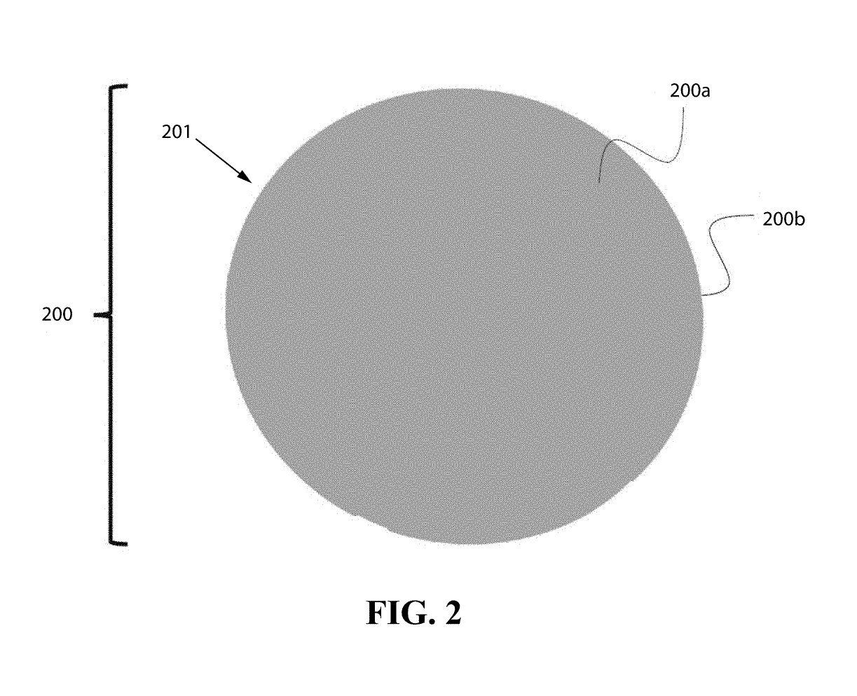

With further reference to the drawings, FIG. 2 shows an example artificial manufactured lipophilic molecule sequestration device embodiment 200 including a nanosphere embodiment 201 comprising one or more biocompatible exteriors 200a and one or more lipophilic interiors 200b according to one embodiment. In some embodiments, the biocompatible exterior 200a may not include pores. As such, in some embodiments, diffusion across the exterior surface may serve to allow sequestration of substances. Moreover, in some embodiments, diffusion may facilitate exclusion of specific substances by size, charge, or other attribute of permeability.

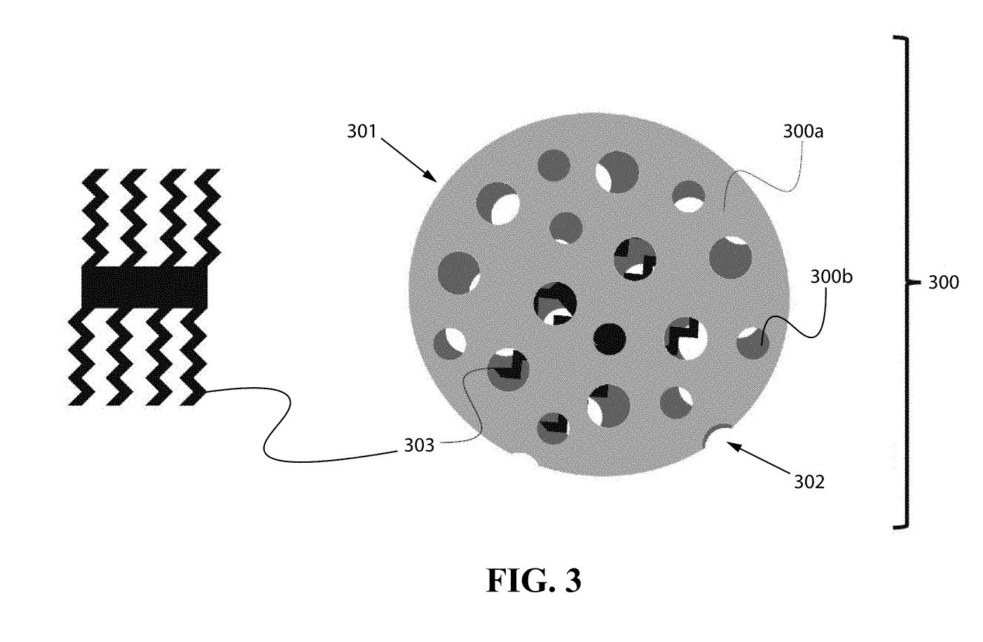

With still further reference to the drawings, FIG. 3 shows an example artificial manufactured lipophilic molecule sequestration device embodiment 300 including a nanosphere embodiment 301 comprising one or more biocompatible exteriors 300a and one or more lipophilic interiors 300b comprising and operable with at least one enclosed resin 303 The term “enclosed resin” as used herein refers to any binding conglomeration that may contain or be made to contain a substance. Substance in some embodiments comprises fatty acids. In some embodiments, an enclosed resin 303 comprises cholestyramine. Cholestyramine is a lipophilic molecule that is known to bind fats and cholesterols. Additionally, in some embodiments, this binding may prevent release of the substances (e.g. causes sequestration). Moreover, in some embodiments, a nanosphere 301 may include one or more pores 302, wherein the one or more pores 302 may be similar in structure and functionality to the pores 102 discussed in relation to FIG. 1. Furthermore, in some embodiments, a biocompatible exterior 300a prevents interaction of the sequestered substances with an environment, such as a lipid-containing environment. Still further, in some embodiments, the enclosed resin 303 may be enclosed by a biocompatible polyethylene glycol exterior by polymerizing polyethylene glycol (PEG) block polymers at 2-100 g/L in aqueous solution under vigorous agitation. In some embodiments, an enclosed resin 303 may comprise enzymes, catalysts, and/or chemical targets that incorporate substances that have been targeted for sequestration. Even further, in some embodiments, inclusion of trapped pyruvate kinase (PK) enzyme may be used to catalyze the sequestration of carbohydrate breakdown products. Such entrapment of materials, in some environments, may be made by restricted pore 302 size. Yet again, in some embodiments, such entrapment may be effectuated by covalent docking. In still other embodiments, inclusion of bound Acetyl CoA:ACP transacylase, Malonyl CoA:ACP transacylase, 3-ketoacyl-ACP synthase, 3-ketoacyl-ACP reductase, 3-Hydroxyacyl ACP dehydrase, and/or Enoyl-ACP reductase enzymes may be used to catalyze the sequestration of fatty acid chain substrates. According to still other embodiments, inclusion of radical-containing lipids may act to further consolidate sequestered lipids by chemical reaction. Such chemical targets may have the advantage of tightly sequestering targeted substances. In still other embodiments, inclusion of alcohol sidechains in the interior may promote esterification to similar effect. Such alcohol sidechains may include glycerol, tyrosine, sugars, or other similar compounds. Still additional embodiments may include bacteria, such as, for example, Akkermansia muciniphila, that consume fats. In such embodiments, the bacteria may be trapped within the interior of the nanocapsules 301 with, for instance, pore 302 sizes under 100 Å that prevent bacterial egress. Such bacteria may act to contain fats by consumption.

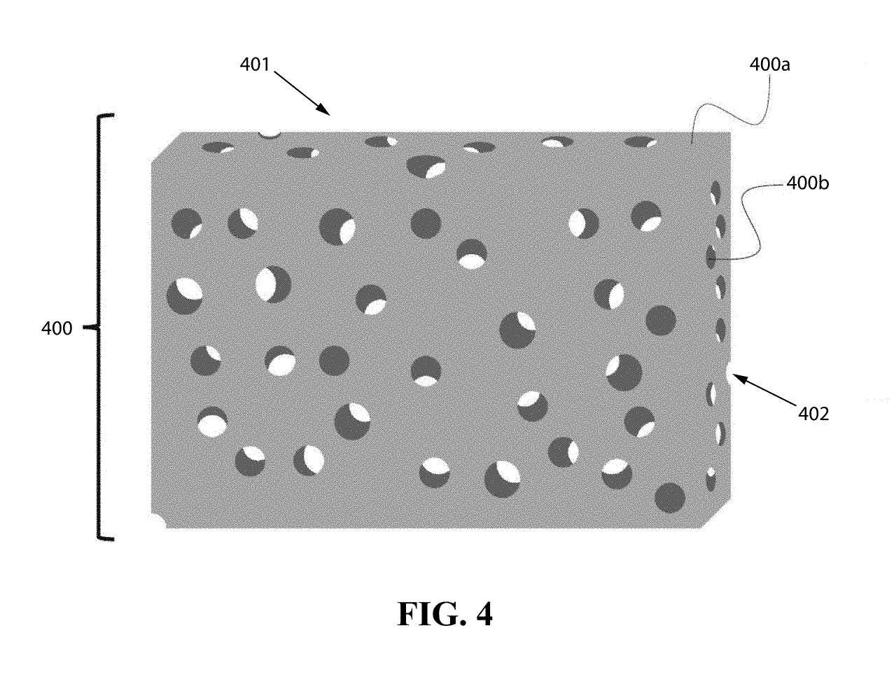

With continued reference to the drawings, FIG. 4 shows an example artificial manufactured lipophilic molecule sequestration device embodiment 400 including a nanosphere 401 comprising one or more biocompatible exteriors 400a and one or more lipophilic interiors 400b according to one embodiment. In some embodiments, a nanosphere 401 may refer to a globular object that may contain or be made to contain a substance. Moreover, in some embodiments, the nanospheres 401 are formed from an amalgamation. This may have the distinct advantages of improving solubility, preventing breakdown, allowing formation into pill form, and/or simplifying manufacture. Furthermore, in some embodiments, the disclosed devices comprising nanocapsules (e.g. nanocapsules, nanodevices, etc) 401 may be added to foods or drinks for consumption into a gastrointestinal environment. A “gastrointestinal environment,” “gastrointestinal tract,” or similar term as used herein refers to one or more animal (including human) gastrointestinal tracts, bacteria, other organisms, organs, organ systems, cells, tissues, nutrients, foods, consumable liquids, feces, urine, or other factors as known by those of ordinary skill in the art. These terms are sometimes used interchangeably in this disclosure and should not be construed as limiting. A gastrointestinal environment may be a lipid-containing environment. In some embodiments, the disclosed artificial manufactured lipophilic molecule sequestration devices 400 may be ingested or otherwise introduced (e.g. by enema, suppository, injection, gavage, or other method known to those skilled in the art) into a gastrointestinal environment with or without other substances.

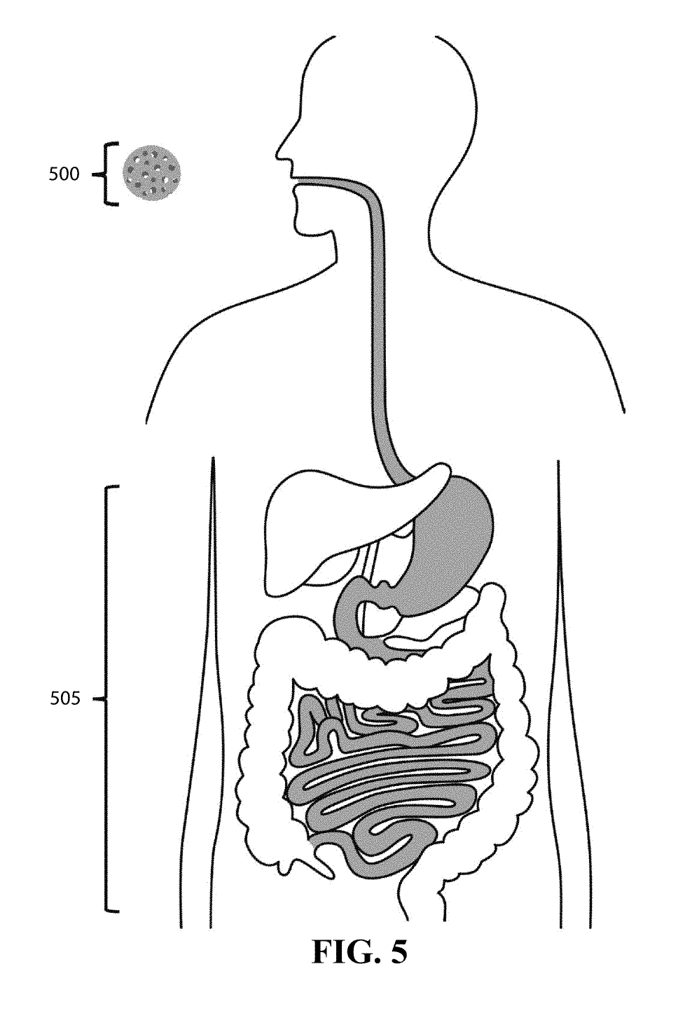

Further disclosure, with reference to the drawings, is set forth in FIG. 5, which shows an example system in which an artificial manufactured lipophilic molecule sequestration embodiment 500 is introduced into a body (such as by being ingested, by oral gavage, by injection, by suppository, by enema, etc.) and passes through an environment, such as gastrointestinal environment 505. It is understood by those of ordinary skill in the art that FIG. 5 is illustrative only and, accordingly, may not be to scale. According to some embodiments, the disclosed ingested or introduced devices 500 are not absorbed by the gastrointestinal tract 505. Further, in some embodiments of ingested or introduced artificial manufactured lipophilic molecule sequestration device embodiments 500 may prevent absorption or interaction of contained (e.g. sequestered) molecules by/with the gastrointestinal environment 505 and/or by environmental microbes.

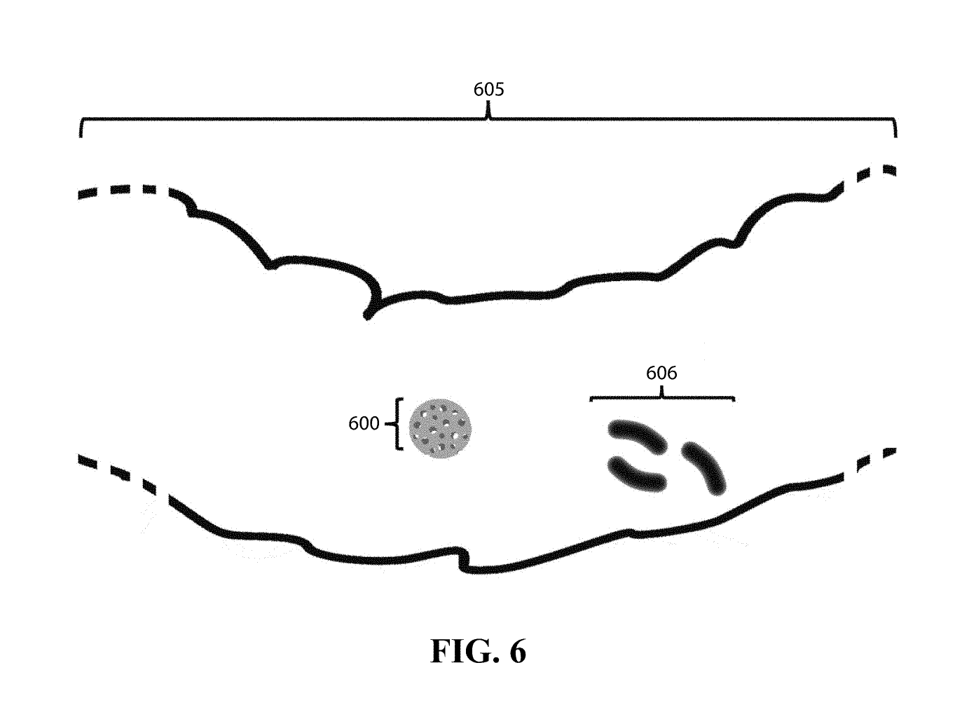

Turning again to the drawings, FIG. 6 shows an example system in which an ingested or otherwise introduced artificial manufactured lipophilic molecule sequestration device embodiment 600 prevents the interaction of sequestered materials with a gastrointestinal environment 605 comprising at least a portion of a gastrointestinal tract with or without bacteria 606. In some embodiments, prevention of interaction of sequestered materials (e.g. fatty acids, fats, etc.) with the gastrointestinal environment 605 prevents the uptake of the sequestered materials into the bloodstream. Additionally, in some embodiments, the sequestered material may comprise fatty acids or fats. Prevention of fatty acid or fat uptake by the gastrointestinal tract into the bloodstream may have the effect of reducing caloric intake, thereby reducing weight gain. Moreover, in some of these embodiments, the sequestered material may comprise sugars. Prevention of sugar uptake in the bloodstream may reduce caloric intake. In some of these embodiments, the sequestered material may comprise toxins. Prevention of toxin uptake in the bloodstream may reduce toxicity.

In some embodiments, prevention of interaction of sequestered materials with a gastrointestinal environment 605 may prevent the materials from building up freely in the gastrointestinal tract. In some of these embodiments, the sequestered material may comprise fatty acids or fats. Prevention of fatty acid or fat buildup in the gastrointestinal environment by such embodiments may provide the unexpected benefit of preventing greasing, lubricating, oiling, or otherwise modifying the gastrointestinal tract, reducing or eliminating the potential side effects of steatorrhea and related conditions including but not limited to anal leakage, gas, diarrhea, bloating, and/or abdominal pain.

Referring further to FIG. 6, in some embodiments, prevention of interaction of sequestered materials with bacteria 606 may have the benefit of preventing the use of sequestered materials by said bacteria. Moreover, in some embodiments, the sequestered material may comprise fatty acids or fats. Additionally, in other embodiments, the sequestered material may comprise sugars. In still other embodiments, the sequestered material may comprise proteins. Prevention of bacterial access to fats or other nutritive substances like proteins, salts, vitamins, or sugars may have the unexpected benefit of preventing bacterial overgrowth and sequelae such as bowel dysmotility, malabsorption, fermentation, and gas production as well as other effects.

Mice were fed artificial manufactured lipophilic molecule sequestration device embodiments over the course of 3 weeks in addition to a high fat diet. Control mice on this same high fat diet gained substantial weight over this period. In contrast, mice fed artificial manufactured lipophilic molecule sequestration device embodiments over the same period lost weight. These mice did not experience observable sequelae such as bowel dysmotility, gas production, diarrhea, or hair loss.

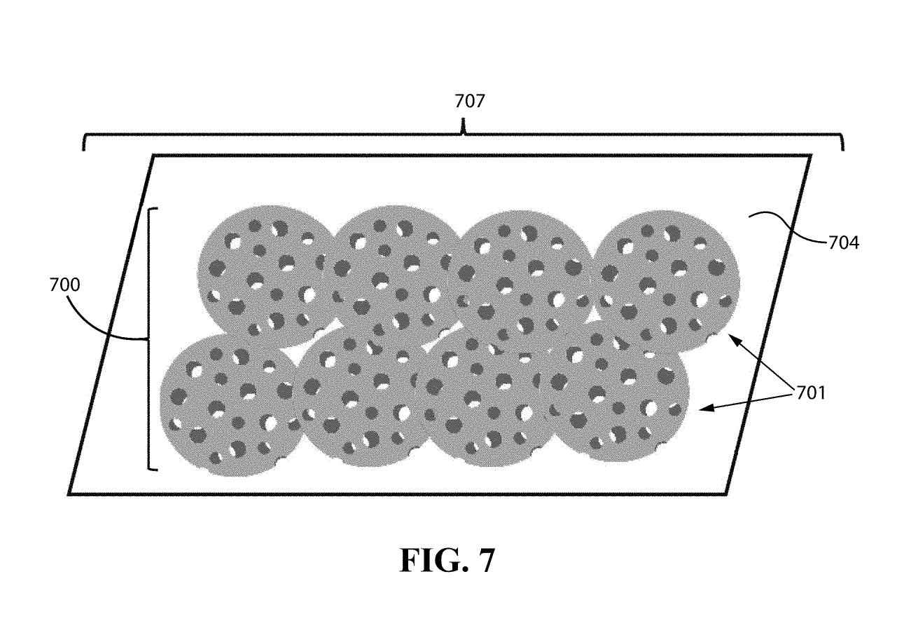

With continued reference to the drawings, FIG. 7 shows an example embodiment of an environment 707 in which ingested or otherwise introduced artificial manufactured lipophilic molecule sequestration device embodiments 700 are passed into fecal matter 704. In some embodiments, sequestered materials may, by virtue of sequestration in a lipophilic interior, be precluded from interacting with the fecal environment 707, which may include compost, bacteria-containing materials, water systems, air, and homes. Unexpected benefits from precluding this interaction may include improved odors, reduced bacterial burden, improved plumbing egress, and/or enhanced sanitation.

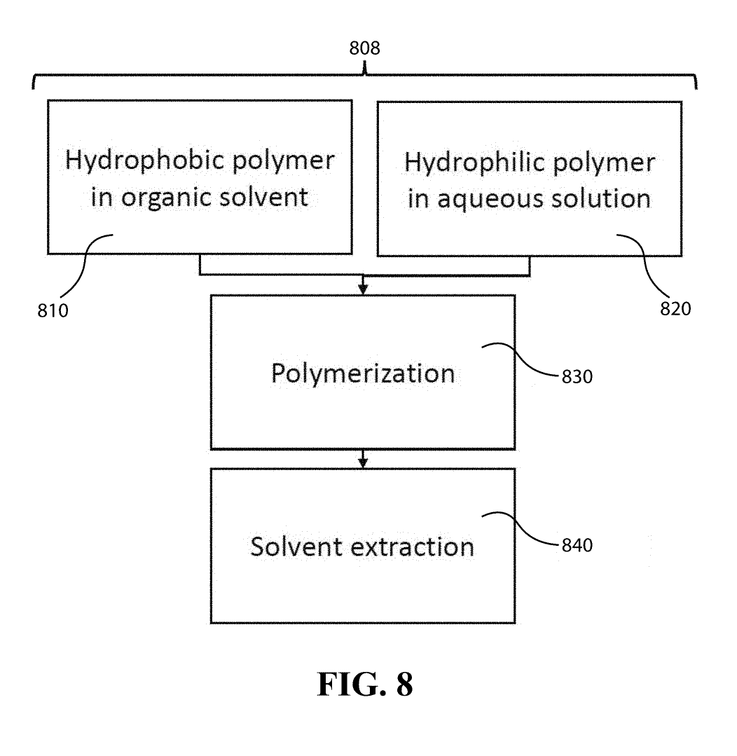

An illustrative schematic of an embodiment of a method of manufacture for producing one or more artificial manufactured lipophilic molecule sequestration devices 808 is depicted in FIG. 8. This illustrative schematic conveys a number of exemplary methods of manufacture of the present disclosure to those of ordinary skill in the art.

According to some embodiments, aspects of methodology for producing one or more artificial manufactured lipophilic molecule sequestration devices 808 may include dissolving a hydrophobic polymer in an organic solvent 810. Another methodological aspect pertaining to the production of one or more artificial manufactured lipophilic molecule sequestration devices 808 may include dissolving a hydrophilic polymer in an aqueous solution 820. In some embodiments, these solutions 810 and/or 820 may further include emulsions, stabilizers, soaps, and/or other additional chemicals. Some of these further included compounds and chemicals may have the unexpected result of improving porosity of the final product. Moreover, some of these compounds and chemicals may have the unexpected result of improving solubility of the solute in the solvent. Still more of these compounds, chemicals and/or corresponding chemical combinations may have the unexpected result of stabilizing various resultant solutions, mixtures, compounds, polymers, and/or pouches. These compounds, chemicals and/or chemical combinations may be referred to as stabilizing agents. Additionally, some of these compounds, chemicals and/or chemical combinations may have the unexpected result of acting as catalysts or cross-linking or polymerizing agents to initiate cross-linking. “Polymerizing” and related terms as used herein such as “cross-linking” refer to the process of joining multiple substances by covalent, ionic, polar, or hydrogen bonding mechanisms. According to some embodiments, heating the solutions 810 and/or 820 may also catalyze cross-linking or polymerization. The aforementioned terminology and associated chemical reactivity, particularly with respect to polymerizing, cross-linking, catalyzing, and otherwise forming, manipulating and utilizing applicable solutions should be known to those with ordinary skill in the art.

According to some embodiments, as further outlined in FIG. 8, organic solution phase 810 and aqueous solution phase 820 may be mixed together to facilitate polymerization 830 of polymers from both solution phases 810 and 820. According to some embodiments, during polymerization, these phases may be vigorously mixed, stirred, vortexed, and/or sonicated, etc., to produce appropriate dispersion. Such dispersive actions may have the unexpected benefit of producing more homogeneous final products. Moreover, the dispersive actions may further have the unexpected benefit of increasing product porosity. During polymerization, one or more subunits of one or more polymers may be polymerized to produce one or more biocompatible exteriors, such as, for example, biocompatible exteriors 100a, 200a, 300a and 400a. In addition, during polymerization, one or more subunits of one or more polymers may be polymerized to produce one or more lipophilic interiors, such as, for example, lipophilic interiors 100b, 200b, 300b and 400b.

Referring still further to FIG. 8., according to some embodiments, another aspect of methodology for producing one or more artificial manufactured lipophilic molecule sequestration devices may include extracting a solvent 840. The solvent, such as an organic solvent, may, inter alia, be extracted by liquid-liquid extraction (LLE), also known as solvent extraction and partitioning, to separate the applicable compounds based on their relative solubilities in two different liquids, thereby extracting a substance from one liquid into another liquid phase. Additionally, solvent extraction 840 may also be effectuated by heat, exchange, or other means, and/or a combination of extraction processes. Solvent extraction 840 may have the unexpected result of improving the porosity of the final product. According to some embodiments of a method of manufacture for producing one or more artificial manufactured lipophilic molecule sequestration devices 808, hydrophobic and hydrophilic polymers may represent the self-same polymer, parts of multiple polymers, and/or non-polymeric substances.

According to one embodiment, a hydrophilic polymer may be ethylene dimethacrylate (EGDMA) or water-soluble polymer. This may be dissolved in water at 0.1-10% w/v according to one embodiment. According to this embodiment, EGDMA may be made more water soluble by adding a detergent, such as, for example, sodium dodecyl sulfate (synonymously sodium lauryl sulfate or laurilsulfate; SDS or SLS, respectively). Such a detergent may improve the water solubility of EGDMA and inter-phase mixing between aqueous and organic phases. According to this embodiment, sodium nitrite (NaNO₂) may be added as a stabilizer.

According to one embodiment, a hydrophobic polymer may be glycidyl methacrylate. In this embodiment, isooctane (also known as 2,2,4-Trimethylpentane) may be added and may have the unexpected effect of improving porosity of the product. Moreover, in this embodiment, 4-methyl-2-pentanol (also known as methyl isobutyl carbinol or MIBC) may be the organic solvent. The organic compound 4-methyl-2-pentanol may have the added benefit of evaporating easily to produce improved porosity of the product. Additionally, in this embodiment, benzoyl peroxide (BPO) may be added to the organic solution and may act as a catalyst for polymerization.

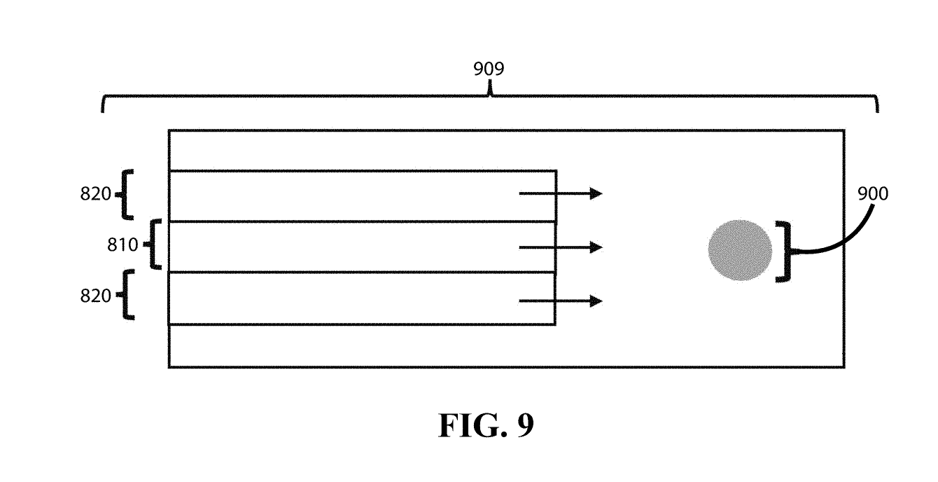

With further regard to the drawings, in some embodiments as shown in FIG. 9, when producing one or more artificial manufactured lipophilic molecule sequestration devices, multiple phases, such as, for example, solution phases 810 and 820 described with respect to FIG. 8 above, may be injected together 909, wherein in such injection may be facilitated, at least in part, by laminar flow. The phases, such as solution phases 810 and 820, may be fully miscible, at least partially miscible, or immiscible, when injected together 909. This methodological process of injecting the phases together 909 may have the unexpected benefit of controlling particle size during extraction. It may also have the unexpected benefit of producing fully homogenous or at least partially homogenous products. In some embodiments, the organic phase, such as organic solvent solution 810, may be injected into the aqueous phase, such as aqueous solution 820, at pressures exceeding 200 mm Hg. This may be performed using an injection device (not shown), as should be understood by those of ordinary skill in the art. This high-pressure liquid-liquid injection mixing may have the unexpected effect of producing small particles with increased porosity. This may further have the unexpected effect of producing particles with pores between 7 Å and 100 Å, which may have the effects of allowing lipids to penetrate the exterior of an artificial manufactured lipophilic molecule sequestration device, such as embodied device 900, while preventing bacteria or other gastrointestinal environmental factors from penetrating the exterior of the sequestration device embodiment 900. A mixture of multiple phases, such as, for example, solution phases 810 and 820, may be warmed in order to evaporate or speed evaporation of a solvent, such as, for example, a dichloromethane solvent. This warming may be accomplished at greater than 30° C. Warming may have the unexpected result of increasing the porosity of the resultant products.

Embodiments such as the aforementioned have been produced using various methods disclosed herein and tested for their ability to sequester fats. In some tests, some embodiments were mixed with fat-soluble dye Oil Red 0 and extracted by centrifugation. Oil Red 0 remaining in solution versus the centrifuge pellet was detected in a colorimetric assay at or around 360 nm. In these tests, Oil Red 0 was sequestered effectively by the tested embodiments. This showed that these embodiments do indeed sequester fat soluble molecules efficiently.

According to some embodiments, the volume of organic solvent solution, such as solution 810, may be kept to less than ⅔ the volume of aqueous solution, such as solution 820, which may have the unexpected result of better internalization of the lipophilic portion of the resultant manufactured and artificial lipophilic molecule sequestration device(s), such as embodied device 900.

Some embodiments of artificial manufactured lipophilic molecule sequestration devices may be manufactured with one or more polymers. According to one embodiment, a combined hydrophilic and hydrophobic polymer may be cyclodextrin or other cyclical sugar polymer. This may be dissolved in 0.1 to 2M NaOH at 2-50% w/v a according to one embodiment. According to this embodiment, a catalyst and/or linker may be ethylene glycol diglycidyl ether (EGDE). This may be added in an aqueous solution at 10-90% w/v. The aforementioned aqueous solutions of cyclodextrin and EGDE may be mixed together. This combined solution may then be heated for 15 to 600 minutes at 30-99° C. to initiate cross-linking. According to this embodiment, an organic phase of dichloromethane may be mixed with sorbitane monooleate as a stabilizing agent. According to this embodiment, both aqueous and organic phases (e.g. cyclodextrin/EGDE and sorbitane monooleate solutions) may be combined. In some embodiments, for example 1 to 50 ml of total aqueous phase may be added to 2 to 100 ml of the organic phase and vigorously mixed. Such a mixture may be warmed to above 25° C. in order to evaporate and extract the dichloromethane solvent. This may have the unexpected result of modulating the porosity of the resultant products.

Many additional embodiments of artificial manufactured lipophilic molecule sequestration devices may be achieved through the use of different combinations of hydrophobic, hydrophilic, and/or amphiphilic polymers. In some embodiments, hydrophobic polymers may include lactide, polylactide, polydimethylsiloxane (PDMS), polycaprolactone (PCL), lactone, polymethylmethacrylate (PMMA), divinyl benzene, polypropylene, other polysaccharide, polypeptides, and/or other suitable polymer or polymerizable substance. In some embodiments, hydrophilic polymers may include polyethylene glycol (PEG), polyethylene oxide (PEO), poly-2-methyloxazoline (PMOXA), polyacrylamide (PAM), polyvinyl alcohol, polyvinylbenzene sulfonic acid, ethyl cellulose, other polysaccharide, polypeptides, algenate, methacrylates, polyurethanes, or other suitable polymer or polymerizable substance. In some embodiments, amphiphilic polymers may include cyclodextrin, cylclical sugar polymers, polyethylene glycol-polypropylene glycol (PEG-PPG), PEO-PPO, PEG diblock, PEG triblock, other suitable polymers, or other suitable block copolymers.

According to some embodiments, hydrophobic cores may be produced by phase separation or coacervation. Coacervation, as understood by those with ordinary skill in the art, refers to composition formation through weak hydrophobic forces and/or covalent bonds. Emulsifiers such as lecithin and polysorbate may further act as stabilizers of the composition. According to some embodiments, pre-formed polymers may be combined with stabilizers to produce suitable nanocapsules. In some embodiments, no polymerization may be necessary. Some embodiments are formed by removal or breakdown of a core mold object. In one example embodiment, carbon is deposited on the surface of a mold object such as a hydrophobic or organic solvent droplet.

According to another embodiment produced by coacervation, a hydrophobic polymer may be polycaprolactone (PCL). According to this embodiment, polysorbate 80, sorbitan monostearate, caprylic/capric triglyceride, and lecithim may be used as surfactants. According to one embodiment, 0.2 to 12 g PCL may be dissolved in 500 ml acetone with 1-10 g sorbitan monostearate and 0.5-50 g caprylic/capric triglyceride. According to this embodiment, more than 2 ml water and 2 ml ethanol may be used to dissolve 0.1 to 5 g lecithin and subsequently added to the above mixture under medium agitation at 30-80° C. or above. This mixture may then be depressurized to remove acetone with a rotary evaporator.

According to some embodiments, hydrophobic cores may be coated with biocompatible substances. This may have the benefit of providing similar effects as dietary fiber. According to one embodiment, hydrophobic cores may be coated with chitosan. According to one embodiment, a chitosan solution may be prepared by dissolving 0.1 to 5 g in 99 ml water, 1 ml glacial acetic acid. The solution may be added slowly under mild agitation for over 2 hours at room temperature. In some embodiments, this mild agitation may include stirring on a magnetic stirrer at less than 200 rotations per minute. In some embodiments, a more vigorous agitation may include stirring in a bladed blender at greater than 200 turns per minute. According to another embodiment, hydrophobic cores may be coated with polymerized biocompatible substances such as PEG and PEO as detailed above.

Some embodiments may be produced by melt dispersion. The dispersion of an alcoholic solution of isobutylcyanoacrylate and oil in water, by interfacial polymerization, may allow the formation of nanocapsules, such as, for example, depicted nanospheres 101, 201, 301, 401, and 701, associated with artificial manufactured lipophilic molecule sequestration devices, such as, for example, embodied devices 100, 200, 300, 400, 500, 600, 700 and 900, wherein the formed nanocapsules may have an average diameter of about 200 nm to 1 cm. Corresponding physical and technical parameters may be studied and determined: for example, temperature of preparation, pH of aqueous phase, concentration of surfactant and ethanol. The determined physical and technical parameters may be investigated with different active molecules and particularly with a radiological tracer, wherein nanocapsule manufacture presents some advantages: (i) preparation may be easily transposable to an industrial scale; and (ii) the method may allow for a high level of entrapment for lipophilic substances.

Replacement fats, in contrast to the present disclosure, often attempt to replace naturally-occurring, digestible dietary fats with indigestible substitutes. Unfortunately, common replacement fats may cause major side-effects such as diarrhea, anal leakage, steatorrhea, bloating, abdominal pain, gas, foul-smelling stools, and bacterial overgrowth. These side-effects are direct consequences of interaction of these fats with an environment such as the gastrointestinal tract. This environment may be a lipid-containing environment and may include the gastrointestinal tract itself and the greasing nature of fats. This environment may also include bacteria and/or other organisms and their ability to alternatively digest undigested or replacement fats. Additional drawbacks of replacement fats include fat-soluble vitamin leaching and the requirement that replacement fats be used in food preparation. Replacement fats are not easily incorporated into existing foods; further, food preparation with replacement fats is, by definition, an industrial process that has been linked with poorer health outcomes. Embodiments of the present disclosure may have the important benefits of: (1) preventing interaction of dietary fats with the gastrointestinal tract or environment, thus avoiding associated side effects as detailed herein and otherwise as known to those of ordinary skill in the art; (2) preventing interaction of dietary fats with bacteria and/or other organisms, thus avoiding associated negative side effects as detailed herein and otherwise as known to those of ordinary skill in the art; and (3) preventing fat digestion, whatever its source, meaning embodiments need not be included in food preparation or processing, thereby avoiding associated negative effects as detailed herein and otherwise as known to those of ordinary skill in the art.

Inhibition of fat digestion and absorption is another method attempted to restrict fat calories. Resins, for instance, suffer from similar drawbacks as replacement fats (e.g. diarrhea, anal leakage, steatorrhea, bloating, abdominal pain, gas, and bacterial overgrowth) as well as from medication side-effects such as drug-drug interactions, cholelithiasis, liver failure, kidney stones, rashes, and drug uptake modulation, and others. These drawbacks occur because inhibitors of fat digestion and resins commonly allow and/or promote interaction of fats with the environment, including but not limited to a gastrointestinal tract and/or bacteria and/or other organisms. The present disclosure has the important benefits as listed above, as well as the benefits of localized effect rather than systemic absorption. In some embodiments, the artificial manufactures lipophilic molecule sequestration devices may be configured such that they are not absorbed into the bloodstream. This benefit may be conferred in some embodiments, unexpectedly, by increased size of the devices (e.g. embodiments greater than 1 μm in overall diameter may be less absorbable in a gastrointestinal tract) or by reduced susceptibility to degradation (e.g. embodiments containing non-hydrolyzable inter-polymer bonds may be less absorbable in a gastrointestinal tract).

Additional benefits of some embodiments of the present disclosure include solubility in water and/or lack of flavor, making the embodiments more palatable and mixable with food, drink, or other substance. Furthermore, certain embodiments may have a number of unexpected technical advantages. For example, benefits of some embodiments may include a reduction in metabolic syndromes, diseases, symptoms, and disease states including but not limited to diabetes, hyperlipidemia, hypercholesterolemia, obesity, elevated blood glucose, peripheral vascular disease, heart disease, claudication, myocardial infarction, thromboembolic events, strokes, lipodystrophy, and other disease states. Certain embodiments may help in treatment of various disease states including but not limited to acute or chronic pancreatitis, pancreatic cancers, pancreatic insufficiencies, malabsorptive states, anomalous gastrointestinal tracts, cholecystitis, renal stones, biliary disease, and genetic syndromes.

Importantly, unexpected benefits of some embodiments disclosed herein may be accomplished without major side-effects including but not limited to diarrhea, steatorrhea, bloating, abdominal pain, gas, bacterial overgrowth, and anal leakage. Other additional potential benefits of some embodiments may include but are not limited to increased exercise tolerance, weight loss, improved psychosocial functioning, and improved functioning in activities of daily living. Some further embodiments may be designed to improve nutrition, augment health, or increase wellbeing or comfort.

According to some embodiments, fat-soluble vitamins or other nutrients such as vitamin A and other carotenoids, vitamin D, vitamin E, vitamin K, may be added in order to replace absorbed nutrients. According to some embodiments, this may include exchangeable nutrients found within the lipophilic interior itself. According to other embodiments, this may include supplemented nutrients not contained in the lipophilic interior. Moreover, according to some embodiments, probiotics such as lactobacilli or other bacteria may be supplemented in order to promote appropriate gut symbiosis. According to still other embodiments, fibers may be supplemented in order to promote appropriate gut motility. In addition, according to still other embodiments, degradation times may be coordinated to improve environmental compatibility.

None of the above should be considered limiting and are disclosed for illustrative purposes only. Although the above descriptions include a number of specific applications, these should not be considered limiting. Various techniques may be used in different contexts, and various contexts may benefit from different techniques and embodiments. Thus, a number of variations may be applied without departing from the scope of the present disclosure. In addition, not all applications are presented as embodiments here. While this disclosure has been described in conjunction with the specific embodiments outlined above, it is evident that many alternatives, modifications and variations will be apparent to those skilled in the art. Accordingly, the preferred embodiments of the present disclosure as set forth above are intended to be illustrative, not limiting. Various changes may be made without departing from the spirit and scope of the present disclosure, as required by the corresponding claims. The claims provide the scope of the coverage of the present disclosure and should not be limited to the specific examples provided herein. Thus the scope of the disclosure should be evaluated according to the appended claims.

1. An artificial manufactured lipophilic molecule sequestration device comprising:
one or more biocompatible exteriors, and
one or more lipophilic interiors;
wherein the artificial manufactured lipophilic molecule sequestration device is configured for introduction into a gastrointestinal environment.

2. The device of claim 1, wherein the artificial manufactured lipophilic molecule sequestration device is configured to sequester at least one lipid molecule from the gastrointestinal environment.

3. The device of claim 1, wherein the biocompatible exteriors comprise hydrophilic molecules.

4. The device of claim 1, wherein the lipophilic interior includes one or more hydrophobic resins.

5. The device of claim 1, wherein the artificial manufactured lipophilic molecule device includes one or more pores.

6. The device of claim 5, wherein at least one of the one or more pores in the artificial manufactured lipophilic molecule device comprise a diameter greater than 7 angstroms and smaller than 100 angstroms.

7. A method for manufacturing an artificial lipophilic molecule sequestration device, the method comprising:
combining one or more polymers in one or more solvents;
adding polymerizing agents; and
producing at least one artificial lipophilic molecule sequestration device;
wherein the at least one artificial lipophilic molecule sequestration device is configured to sequester lipids from a gastrointestinal environment.

8. The method of claim 7, wherein one or more subunits of one or more polymers are polymerized to produce one or more biocompatible exteriors.

9. The method of claim 8, wherein at least one of the one or more biocompatible exteriors are hydrophilic.

10. The method of claim 7, wherein one or more subunits of one or more polymers are polymerized to produce one or more lipophilic interiors.

11. The method from claim 10, wherein the one or more lipophilic interiors include a hydrophobic resin.

12. The method of claim 7, wherein a solvent is extracted to improve porosity.

13. The method of claim 7, wherein the gastrointestinal environment is a human gastrointestinal tract.

14. The method from claim 7, wherein the at least one artificial manufactured lipophilic molecule sequestration device includes one or more pores that allow entry of lipophilic molecules based on a size of the one or more pores.

15. The method from claim 7, wherein the at least one artificial manufactured lipophilic molecule sequestration device includes one or more pores that exclude bacteria based on a size of the one or more pores.

16. A method for lipid sequestration comprising:
introducing, into a lipid-containing environment, a plurality of artificial manufactured lipophilic molecule sequestration devices, each of the plurality of artificial manufactured lipophilic molecule sequestration devices comprising:
one or more biocompatible exteriors; and
one or more lipophilic interiors;
wherein the plurality of artificial manufactured lipophilic molecule sequestration devices sequester one or more lipophilic molecule(s) to reduce interaction between the one or more lipophilic molecule(s) and the lipid-containing environment.

17. The method from claim 16, wherein the environment comprises a gastrointestinal environment.

18. The method from claim 16, wherein the environment comprises bacteria.

19. The method from claim 16, wherein at least one of the artificial manufactured lipophilic molecule sequestration devices includes one or more pores configured to be sufficiently large to allow entry of fatty acids, and sufficiently small to exclude bacteria.

20. The method from claim 19, wherein one or more pores of the artificial manufactured lipophilic molecule sequestration devices may open, close, or include specificity based on chemical makeup.

21. The method from claim 16, wherein one or more of the plurality of artificial manufactured lipophilic molecule sequestration devices are ingested or otherwise introduced into a body.

22. The method from claim 16, wherein a lipophilic interior includes a hydrophobic resin.

23. A lipid sequestration system comprising:
a plurality of artificial manufactured lipophilic molecule sequestration devices comprising:
one or more biocompatible exteriors, and
one or more lipophilic interiors; and
an environment comprising a plurality of lipophilic molecules;
wherein at least one artificial manufactured lipophilic molecule sequestration device of the plurality of artificial manufactured lipophilic molecule sequestration devices absorbs and sequesters at least one lipophilic molecule of the plurality of lipophilic molecules to reduce interaction with the environment.

24. The system from claim 23, wherein the at least one artificial manufactured lipophilic molecule sequestration device of the plurality of artificial manufactured lipophilic molecule sequestration devices includes one or more pores that are:
sufficiently large to allow entry of fatty acids; and
sufficiently small to exclude bacteria and enzymes.

25. The system from claim 24, wherein one or more pores are smaller than 100 angstroms and larger than 7 angstroms in order to selectively include lipophilic molecules and exclude bacteria.

26. The system from claim 23, wherein the environment comprises a gastrointestinal environment.

27. The system from claim 23, wherein the plurality of artificial manufactured lipophilic molecule sequestration devices is ingested or otherwise introduced into a gastrointestinal environment.
